(19) United States
(12) Patent Application Publication
(10) Pub. No.: US 2016/0135784 A1
(43) Pub. Date: May 19, 2016

(54) Ultrasound Probe Cover

(71) Applicant: Morteza Gharib, Hollidaysburg, PA (US)

(72) Inventor: Morteza Gharib, Hollidaysburg, PA (US)

(21) Appl. No.: 14/542,623

(22) Filed: Nov. 16, 2014

(51) Int. Cl. A61B 8/00

(52) U.S. Cl. CPC A61B 8/4422; A61B 8/4281

(57) ABSTRACT

A new and improved sterile ultrasound probe cover, said cover comprising two layers. The inner layer being placed inside the outer layer, the cover is gelled inside both inner and outer layers. The present invention can be manufactured inexpensively, is easy to use, and can cover an ultrasound probe in sterile manner very quickly, in amount of time which is appealing to practitioners and can be used without using an assistant.

### CROSS-REFERENCE TO RELATED APPLICATION

This application claims the benefit of U.S. provisional patent application No. 61/906,116 filed on Nov. 19, 2013, titled “Ultrasound Probe Cover,” the disclosure of which is herein incorporated by reference.

### STATEMENT REGARDING FEDERALLY SPONSORED RESEARCH

Not applicable.

### FIELD OF THE INVENTION

The present invention relates to medical devices. More particularly, the invention relates to sterile covers for covering an unsterile ultrasound probe cover.

### BACKGROUND DESCRIPTION

An ultrasound device is an imaging device used to visualize the underlying body tissues of a patient for variety of medical procedures. It generally has an ultrasound emitting/receiving probe and an image display monitor. The probe itself consists of a head, which contains ultrasound emitting crystals, and a base. In wired ultrasound devices, the base of the probe connects to the monitor through a specific electric cable. Usually the base of the probe is held in one's hand in order to place the head over a patient's body.

A majority of procedures which require ultrasound imaging are done under sterile techniques. In order to maintain sterility of the surgical field, the un-sterile probe has to be covered with a sterile cover. There are a variety of different sterile covers available in the market. Some are longer to cover the entire probe and its cable, while others are shorter to cover only the probe itself. These covers generally are bulky and relatively difficult to use. In order to cover the probe, the practitioner needs to wear sterile gloves, remove the probe cover from its packaging, and hold the sterile cover in a certain position, ask an assistant to apply ultrasound gel inside the cover, and insert the un-sterile probe inside the sterile cover. The assistant is needed because one cannot touch an unsterile probe and sterile probe cover simultaneously without violating the sterility of the probe cover. Next, a quantity of sterile ultrasound gel is applied to the skin to be imaged and then the covered probe can be placed over a patient area in a sterile manner. As it can be realized, the process of covering a probe with sterile cover and applying two volumes of gel can be inconvenient and relatively time consuming, especially when an assistant is needed but not readily available.

In the practice and administration of anesthesia and pain medicine, ultrasound devices are frequently used to inject local anesthetics in close proximity of a nerve or a nerve bundle. Sometimes a needle is used to inject the local anesthetic once. This is called a single shot technique as opposed to a continuous regional anesthesia technique which uses an indwelling catheter to deliver local anesthetics over a period of few days.

In a busy anesthesia and pain-management practice, placing a single shot block may take only few minutes. It is recommended that the practitioner use complete sterility precautions during regional anesthesia techniques. As mentioned earlier, covering a probe with sterile cover is a two-person and relatively time consuming process. In order to save time, and avoid using an assistant which may not be readily available, sometimes practitioners skip this important step and try to sterilize only the needle insertion site of the skin. This is sometimes called a clean technique, where the practitioner will not use a sterile probe cover, but instead the un-covered probe is placed over the patient's skin close to the needle insertion site. Then the skin next to the probe is prepped with disinfectant solution and a needle is placed into the skin, while the probe is maintained in close proximity and away from the insertion site. It is important to keep the probe away and not contaminate the insertion site or the needle with the unsterile probe, a task which is not always done easily and correctly. The probe head which is covered with ultrasound gel can and frequently will slide into the sterile field and may contaminate the injection site and the needle. More importantly the probe frequently comes in contact with patient's blood which may transfer pathogens to the next patient if the probe is not cleaned and disinfected properly.

Several probe covers are described in the prior art, which all tried in different ways to simplify the practice of applying a sterile cover to an ultrasound probe.

For example, U.S. Pat. Nos. 5,795,632 and 7,665,893 to Buchalter describe pre-gelled probe covers which are placed inside a wrapper. The cover is joined to the wrapper by a layer of adhesive which needs to be peeled off to expose the cover. The cover is intended for specifically shaped endocavity probes. It has been described that a user can hold the probe with one hand and insert the probe inside the cover with the other hand. This cover is a non-sterile probe cover and is designed to keep the probe clean in non-sterile procedures. Should this probe cover be sterilized, and to maintain sterility, an assistant should be used to place the unsterile probe inside sterile cover. In addition, a separate packet of sterile gel should be provided to be applied to the patient body.

In U.S. Pat. No. 5,997,481 to Adams, a sterile pre-gelled probe cover is described. A deformable pocket or reservoir is added to the bottom of the cover. This reservoir can release specific quantity of gel inside the probe to maintain airless contact between the probe and the cover. This invention requires relatively complex and expensive manufacturing process to add the reservoir to the bottom of the probe. Moreover, as it was mentioned earlier, the problem still exists that the practitioner needs to hold the probe in specific position for the assistant to insert the probe inside the cover.

Patent application 20100234733 to Wahlheim describes a similar product in which a sterile probe cover is described with sealed pouches that upon pressure can rupture and release ultrasound gels. Again this is a relatively complex and expensively-made product, which again requires inconvenient and time consuming process of two person operations to cover the probe in sterile operations.

U.S. Pat. No. 6,051,293 to Weilandt describes another similar prove cover. The cover is mounted on a separate sheath of paper and has a sealed chamber of ultrasound gel at the bottom. The seal can break upon insertion of an ultrasound probe into the cover. This invention also requires an assistant because the operator cannot touch and manipulate an unsterile probe and the sterile probe cover at the same time. Besides, a separate sterile packet of gel should be provided to be applied to the skin of the patient.

As it can be seen, there is currently a need for a sterile ultrasound probe cover which can be manufactured inexpensively, is easy to use, and can cover the ultrasound probe in sterile manner very quickly in amount of time which is appealing to practitioners and can be used without using an assistant.

### BRIEF DESCRIPTION OF THE DRAWINGS

FIGS. 1-6 are plan views of the probe cover according to the invention.

### SUMMARY OF THE INVENTION

The probe cover of the current invention is made of two separate flexible sheaths, and inner sheath and an outer sheath. The inner sheath is longer than the outer sheath and is placed inside the outer sheath. In certain embodiments of the invention, the extra length of the inner sheath is folded outwardly to at least partially cover the exterior surface of the outer sheath. Two volumes of gel are disposed at the bottom of the inner sheath and the bottom of the outer sheath. A tight clamp is applied at midway along of the length of the two-layer cover to keep the gels from spilling out. Upon placement of the ultrasound probe inside the probe cover, the folded portion of the inner sheath is unfolded and pulled up to cover the rest of the probe and possibly its cable. A plastic clip or adhesive tape is used to secure the inner sheath around the probe or its cable. The outer sheath is then removed and the probe covered with the inner sheath is placed over the desired area to be imaged. No extra coupling gel is needed as the inner cover covering the probe is gelled both at inner and outer surface.

### DETAILED DESCRIPTION OF THE INVENTION

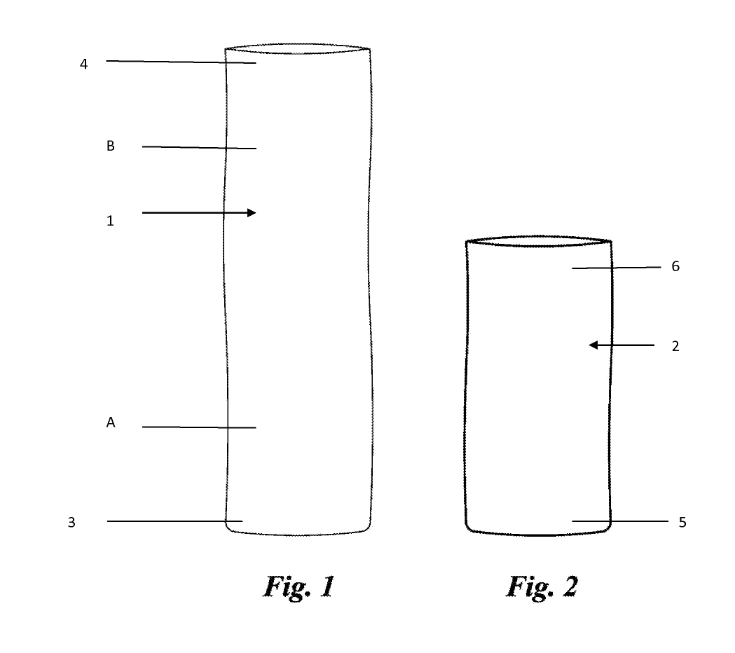

According to FIGS. 1-6, the current invention is comprised of two different sheaths, an inner sheath 1 and an outer sheath 2. The inner sheath 1 has a hollow body having an interior surface and an exterior surface. Said interior surface defines a chamber for receiving the ultrasound probe. The outer sheath 2 has a hollow body having an interior surface and an exterior surface. Said interior surface defines a chamber for receiving the inner sheath 1.

In order to fit most currently and commercially available ultrasound probes, the outer sheath 2 should be about 6-8 centimeter wide and about 8 to 12 centimeter long. It has an open mouth 6 and a closed end 5. The inner sheath 1 is made with almost the same width as the outer sheath 2 but has a length of about 20-24 centimeter. The inner sheath 1 comprises portion A and portion B. Portions A and B are continuous bodies which together make one hollow piece which has a closed end 3 and an open end 4. The inner sheath 1 is manufactured out of a medical grade material which is impermeable to liquids and gel, while allowing the ultrasound signals to pass through. It can be made of thin-walled flexible plastic or latex or any similar material with which conventional probe covers are made. It has to be thin and flexible enough to accept the shape of the probe head and maintain a close contact to it. Generally, a thickness of between 15 to 50 micrometers works for most applications.

The inner sheath 1 has a length to cover the ultrasound probe head and varying degree of the probe body or even its electric cable, based on the shape and size of the probe. The outer sheath 2 can be made of soft and flexible and even semi-flexible, but preferably, it is made of soft plastic material and generally has a thickness of more than the thickness of inner sheath. The outer sheath 2 is impermeable to liquids and ultrasound gels, and will be removed before the ultrasound probe, covered with sterile inner sheath 1, is placed over a patient's body.

At the manufacturing facility, 10-15 ml of coupling gel 8 is placed inside closed end 5 of the outer sheath 2. Portion A of the inner sheath 1 is then inserted inside the outer sheath 2 and advanced all the way down, in such a manner that the inner sheath's 1 closed end 3 is placed inside the outer sheath's 2 closed end 5 and comes in contact with gel 8. Next, 3-5 ml of coupling gel 7 is applied inside the closed end 3 of the outer sheath 2. Portion B of inner sheath 1 is then folded outwardly over the open end 6 of the outer sheath 2 to cover the external surface of outer sheath 2 partially or completely. As it can be realized by this description, the inner sheath 1 which ultimately will be the only sheath to cover the ultrasound probe during image acquisition will be gelled both over the inner surface and the outer surface of the closed end 3. It is gelled inside by gel volume 7 and gelled outside by contact and transfer of gel volume 8. These two layers of gel will serve to provide an airless contact between the probe head and the patient's body.

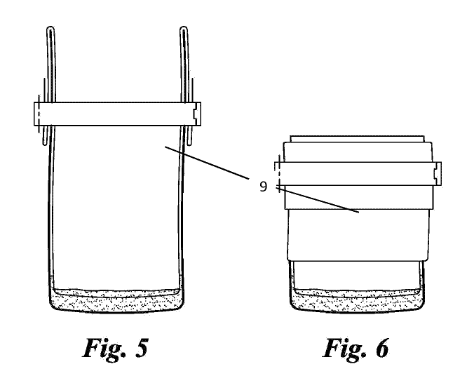

Referring to FIGS. 5 and 6, a clamp 9 is placed over the combined inner and outer sheaths 1, 2. The clamp 9 is placed above the closed ends 3, 5 which contain gels 7, 8. The clamp 9 will maintain gels 7, 8 inside the inner and outer sheaths 1 and 2 and prevent the gels 7, 8 from moving upward and spilling out. The clamp 9 has to be tight and plays a critical role in maintaining the gels 7, 8 inside the bottom ends 3, 5 and maintain sterility and integrity of the gels 7, 8. The two layer probe cover is then packaged and sterilized.

In an alternate embodiment, the two layer probe is folded at a level just above the gel volumes 7, 8. A piece of rubber band or a plastic clamp can be placed tightly over the cover to keep the cover folded and maintain the gels 7, 8 inside the cover and prevent it from spilling out.

The folding of portion B over the outer sheath 2 is critical to cover and protect the open mouth 6. Portion B covers and protects the open mouth 6 of the outer sheath 2 from touching and contamination during handling of the probe cover. If the open end 6 is touched and contaminated, it can render the inner sheath 1 unsterile as the closed end 3 of inner sheath 1 comes in contact with the open end 6 while it is being pulled out of outer sheath 2.

At the patient's bedside and upon opening of the package, the practitioner holds the double sheath probe cover with one hand and removes the clamp 9. Next, using his/her other hand, he or she inserts the ultrasound probe into the inner sheath 1 and pushes the probe all the way into the inner sheath 1 until the probe head comes in close contact with the closed end 3 and gel 7. Then the folded portion B of the inner sheath 1 is unfolded and pulled up to cover the base of the ultrasound probe. The unfolded portion B can be secured around the base of ultrasound probe or its cable, using a piece of adhesive tape, rubber band or a plastic clip. Next, the ultrasound probe, while it is covered by the sterile inner sheath 1 is grabbed at its base and pulled out of the outer sheath 2 and placed over the desired area of a patient's body. At this time, the gel 7 is creating an airless contact between the probe head and the inner sheath 1. The gel 8 is partially transferred to the exterior surface of the closed end 3. This partially transferred gel 8 will create an airless contact between the probe cover and the patient body.

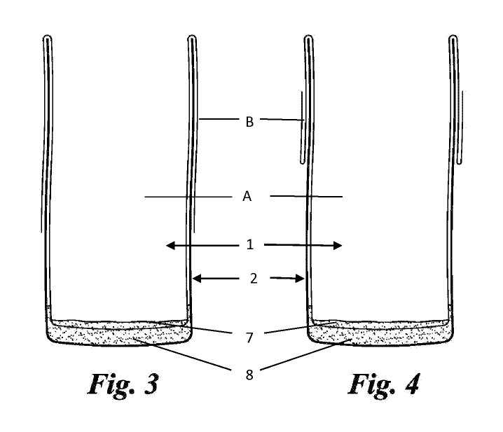

As it can be seen in FIGS. 3 through 6, the outer sheath 2 covers and maintains sterility of the inner sheath 1 as it has been held and manipulated by the practitioner, and allows a practitioner to place an ultrasound probe inside a probe cover very quickly and in a sterile manner without using an assistant. Moreover there is no need for the extra time-consuming process of adding ultrasound gel to the head of ultrasound probe or to the patient's skin.

In handling of this probe cover, the practitioner has the option of donning sterile gloves or not. Even without using sterile gloves, the sterility of the inner sheath 1 is maintained as long as the practitioner holds and touches the ultrasound probe at its base and not the probe head and its sterile cover.

What is claimed is:

1. A probe cover for covering an ultrasound probe, said probe comprising:
An inner sheath, said inner sheath comprising an open mouth, a closed end, an interior surface and an exterior surface, said interior surface defining a chamber for receiving an ultrasound probe, said inner sheath further comprising a portion A, a portion B, and a quantity of ultrasound coupling agent inside said closed end;
An outer sheath, said outer sheath having an open mouth, a closed end, an interior surface and an exterior surface, said interior surface defining a chamber for receiving said inner sheath, said outer sheath further comprising a quantity of ultrasound coupling agent inside said closed end, wherein said portion A being placed inside said outer sheath in a manner that said closed end of said inner sheath comes in close contact with said quantity of coupling agent at said closed end of said outer sheath, said portion B being folded outwardly to cover at least a portion of the exterior surface of said outer sheath; wherein said inner sheath has a length that is greater than
A clamp, said clamp being placed over the combined said inner and outer sheaths in a tight manner to maintain said quantity of coupling agent at the said close ends of said inner and outer sheaths.

2. A method of manufacturing an ultrasound probe cover, said method comprising the steps of:
Providing an inner sheath, said inner sheath having an interior surface, an exterior surface, a portion A, a portion B, an open mouth, and a closed end, wherein said interior surface defines a chamber for receiving an ultrasonic probe;
Providing an outer sheath, said outer sheath having an interior surface, an exterior surface, an open mouth, and a closed end, said interior surface defining a chamber for receiving said inner sheath, wherein said inner sheath is longer than said outer sheath;
Placing a quantity of ultrasound coupling agent to said closed end of said outer sheath;
Placing said portion A of said inner sheath inside said outer sheath in a manner that said closed end of said inner sheath is in close contact with said quantity gel inside said closed end of said outer sheath;
Placing a quantity of ultrasound coupling agent inside said close end of said inner sheath;
Folding said portion B of said inner sheath outwardly to cover at least a portion of said exterior surface of said outer sheath; and
Placing a clamp over the combined said inner sheath and outer sheath in a tight manner to maintain said quantities of coupling agent inside said closed ends of said inner and outer sheath and preventing said agent from spilling out.
